(19) United States
(12) Patent Application Publication
(10) Pub. No.: US 2009/0170205 A1
(43) Pub. Date: Jul. 2, 2009

(54) METHOD OF MAINTENANCE AND EXPANSION OF HEMATOPOIETIC STEM CELLS

(75) Inventors: Hiroyuki MIYOSHI, Tsukuba-shi (JP); Natsumi SHIMIZU, Tsukuba-shi (JP); Hiroaki KODAMA, Wako-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: RIKEN, Wako-shi (JP)

(21) Appl. No.: 12/277,362

(22) Filed: Nov. 25, 2008

(30) Foreign Application Priority Data
Dec. 28, 2007 (JP) 2007-341392

(51) Int. Cl. C12N 15/64; C07K 14/00; C12N 15/63; C12N 5/00

(52) U.S. Cl. 435/455; 530/350; 435/320.1; 435/325; 435/377

(57) ABSTRACT

[Problem] Provided are a method of maintaining/expanding hematopoietic stem cells, a hematopoietic stem cell population obtained by the method, a hematopoietic function ameliorating agent based on administration of the hematopoietic stem cell population to a living organism, and the like.

[Solving Means] A method of maintaining/expanding hematopoietic stem cells, comprising culturing hematopoietic stem cells in the presence of the HSC activity supporting factor of the present invention, which comprises the same or substantially the same amino acid sequence as an amino acid sequence shown by SEQ ID NO:2 or 4, or in the co-presence of a mammalian cell, preferably a stromal cell, incorporating an expression vector harboring a nucleic acid that encodes the HSC activity supporting factor, a cell population containing expanded hematopoietic stem cells obtained by the method, and a hematopoietic function ameliorating agent comprising the cell population.

### TECHNICAL FIELD

The present invention relates to a method of maintaining/expanding hematopoietic stem cells using a protein that supports maintenance/expansion of hematopoietic stem cells, a cell population containing expanded hematopoietic stem cells obtained by the method, a method of improving hematopoietic function using the cell population, and the like.

Possessing self-renewing capacity and multi-lineage differentiation potency, hematopoietic stem cells (HSCs) continue to supply all blood corpuscular cells to individual organisms throughout the life spans thereof. Recent years have seen remarkable advances in hematopoietic stem cell transplantation, a therapy making it possible to restore a normal hematopoietic system by transplantation of hematopoietic stem cells in patients in whom normal production of blood cells is affected by a disease of the hematopoietic system or a cancer treatment. Major modes of hematopoietic stem cell transplantation are bone marrow transplantation, peripheral blood stem cell transplantation, and umbilical cord blood transplantation. In bone marrow transplantation, not only a normal autologous bone marrow, but also a bone marrow from another person with a compatible type of human leukemia antigen (HLA) is transplanted. Because transplantation cannot be performed without HLA type compatibility at a given level or higher, however, there is currently a shortage of donors. Peripheral blood stem cell transplantation necessitates administration of granulocyte colony stimulating factor (G-CSF) to increase hematopoietic stem cells in the donor's peripheral blood, posing a problem with adverse reactions (e.g., pain, increased risks of myocardial infarction and cerebral infarction, and the like). Although umbilical cord blood transplantation is characterized by the minimal physical burden on the donor and a wide range of transplantable HLA types, the availability of umbilical cord blood supply is limited. Therefore, it is considered that if culture conditions that facilitate the ex vivo expansion of HSCs are established, the above-described problems in regenerative medicine utilizing hematopoietic stem cells can be solved.

Although many attempts have been made so far to use a combination of a plurality of cytokines to achieve ex vivo expansion of HSCs, there has been only limited success in a several-fold increase in HSC count even under the optimum culture conditions (non-patent documents 1 and 2); the minimal requirement for practical application has not been reached. Furthermore, a major problem with the ex vivo expansion of hematopoietic stem cells resides in the fact that the self-renewing capacity is lost as a result of promotion of HSC proliferation toward differentiation under conventional culture conditions involving in vitro stimulation of hematopoietic stem cell proliferation with existing cytokines (non-patent documents 3 to 8). Therefore, to achieve ex vivo expansion of HSCs, a further understanding of the mechanisms that regulate the self-renewal and differentiation of HSCs is necessary.

HSCs are thought to be present in a particular microenvironment known as a niche, composed of stromal cells, which play an important role in the determination of the fate of stem cells, in the adult bone marrow. Some stromal cell lines have been established not only from bone marrow, but also from fetal livers and the aorta-gonad-mesonephros region, and have been shown to maintain HSCs in vitro (non-patent documents 9 to 16). Therefore, by co-culturing HSCs with a stromal cell line, a useful system for research into hematopoiesis in niches is provided. However, although it has been demonstrated that physical contact of HSCs and a stromal cell line is essential, little is known about details of the cellular and molecular mechanisms behind the maintenance of HSCs by a stromal cell line.

Meanwhile, MC3T3-G2/PA6 (PA6) is a preadipose stromal cell line derived from the newborn mouse calvaria, and is known to support long-term hematopoiesis in vitro (non-patent document 17). Additionally, three PA6 subclones incapable of supporting the survival of HSCs have been isolated. Furthermore, it is also known that the hematopoiesis-supporting capacity of PA6 cells is not conferred solely by the expression of stem cell factor (SCF), a c-kit ligand, but that some other factor is required. However, nothing has been elucidated so far about the identity of the other factor required for the support of HSC activity.

[Non-patent document 1] G. Sauvageau, N. N. Iscove, and R. K. Humphries, In vitro and in vivo expansion of hematopoietic stem cells, Oncogene 23 (2004) 7223-7232.

[Non-patent document 2] B. P. Sorrentino, Clinical strategies for expansion of haematopoietic stem cells, Nat Rev Immunol 4 (2004) 878-888.

[Non-patent document 3] K. M. Knobel, M. A. McNally, A. E. Berson, D. Rood, K. Chen, L. Kilinski, K. Tran, T. B. Okarma, and J. S. Lebkowski, Long-term reconstitution of mice after ex vivo expansion of bone marrow cells: differential activity of cultured bone marrow and enriched stem cell populations, Exp Hematol 22 (1994) 1227-1235.

[Non-patent document 4] S. O. Peters, E. L. Kittler, H. S. Ramshaw, and P. J. Quesenberry, Murine marrow cells expanded in culture with IL-3, IL-6, IL-11, and SCF acquire an engraftment defect in normal hosts, Exp Hematol 23 (1995) 461-469.

[Non-patent document 5] C. M. Traycoff, K. Cornetta, M. C. Yoder, A. Davidson, and E. F. Srour, Ex vivo expansion of murine hematopoietic progenitor cells generates classes of expanded cells possessing different levels of bone marrow repopulating potential, Exp Hematol 24 (1996) 299-306.

[Non-patent document 6] M. Bhatia, D. Bonnet, U. Kapp, J. C. Wang, B. Murdoch, and J. E. Dick, Quantitative analysis reveals expansion of human hematopoietic repopulating cells after short-term ex vivo culture, J Exp Med 186 (1997) 619-624.

[Non-patent document 7] H. Glimm, I. H. Oh, and C. J. Eaves, Human hematopoietic stem cells stimulated to proliferate in vitro lose engraftment potential during their S/G(2)/M transit and do not reenter G(O), Blood 96 (2000) 4185-4193.

[Non-patent document 8] H. Ema, H. Takano, K. Sudo, and H. Nakauchi, In vitro self-renewal division of hematopoietic stem cells, J Exp Med 192 (2000) 1281-1288.

[Non-patent document 9] L. S. Collins, and K. Dorshkind, A stromal cell line from myeloid long-term bone marrow cultures can support myelopoiesis and B lymphopoiesis, J Immunol 138 (1987) 1082-1087.

[Non-patent document 10] H. J. Sutherland, C. J. Eaves, P. M. Lansdorp, J. D. Thacker, and D. E. Hogge, Differential regulation of primitive human hematopoietic cells in long-term cultures maintained on genetically engineered murine stromal cells, Blood 78 (1991) 666-672.

[Non-patent document 11] C. M. Baum, I. L. Weissman, A. S. Tsukamoto, A. M. Buckle, and B. Peault, Isolation of a candidate human hematopoietic stem-cell population, Proc Natl Acad Sci USA 89 (1992) 2804-2808.

[Non-patent document 12] C. Issaad, L. Croisille, A. Katz, W. Vainchenker, and L. Coulombel, A murine stromal cell line allows the proliferation of very primitive human CD34++/CD38− progenitor cells in long-term cultures and semisolid assays, Blood 81 (1993) 2916-2924.

[Non-patent document 13] H. Kodama, M. Nose, S, Niida, S, Nishikawa, and S, Nishikawa, Involvement of the c-kit receptor in the adhesion of hematopoietic stem cells to stromal cells, Exp Hematol 22 (1994) 979-984.

[Non-patent document 14] K. A. Moore, H. Ema, and I. R. Lemischka, In vitro maintenance of highly purified, transplantable hematopoietic stem cells, Blood 89 (1997) 4337-4347.

[Non-patent document 15] O. Ohneda, C. Fennie, Z. Zheng, C. Donahue, H. La, R. Villacorta, B. Cairns, and L. A. Lasky, Hematopoietic stem cell maintenance and differentiation are supported by embryonic aorta-gonad-mesonephros region-derived endothelium, Blood 92 (1998) 908-919.

[Non-patent document 16] M. J. Xu, K. Tsuji, T. Ueda, Y. S. Mukouyama, T. Hara, F. C. Yang, Y. Ebihara, S. Matsuoka, A. Manabe, A. Kikuchi, M. Ito, A. Miyajima, and T. Nakahata, Stimulation of mouse and human primitive hematopoiesis by murine embryonic aorta-gonad-mesonephros-derived stromal cell lines, Blood 92 (1998) 2032-2040.

[Non-patent document 17] H. A. Kodama, Y. Amagai, H. Koyama, and S. Kasai, A new preadipose cell line derived from newborn mouse calvaria can promote the proliferation of pluripotent hemopoietic stem cells in vitro, J Cell Physiol 112 (1982) 89-95.

### DISCLOSURE OF THE INVENTION

### Problems to be Solved by the Invention

Accordingly, it is an object of the present invention to identify a novel factor required for ex vivo expansion of hematopoietic stem cells in large amounts while maintaining the undifferentiated state thereof, and to provide a method of maintaining/expanding hematopoietic stem cells using the factor. It is another object of the present invention to provide a hematopoietic stem cell population obtained by the method, a hematopoietic function ameliorating agent comprising the hematopoietic stem cell population, and the like.

### Means of Solving the Problems

Hence, the present inventors performed microarray analyses on PA6 cells and PA6 subclone cells lacking hematopoiesis-supporting capacity, and selected 11 genes downregulated specifically in the subclone cells, as candidate genes responsible for the hematopoiesis-supporting capacity of PA6 cells. The present inventors then determined whether or not the capability of supporting the maintenance of the undifferentiated state and expansion of HSCs is restored by introducing and overexpressing a cDNA of each of these genes into the subclone cells. As a result, it was shown that a functionally unknown gene that presumably encodes an endogenous membrane protein, 1110007F12Rik (also called Tmem140), is capable of causing a partial recovery from the lack of HSC-supporting activity in PA6 subclone cells. The present inventors found that the protein thus expressed is useful in the maintenance/expansion of hematopoietic stem cells (the protein is hereinafter also referred to as “the HSC activity supporting factor of the present invention”), and completed the present invention.

Accordingly, the present invention provides:
[1] An agent for maintaining or expanding hematopoietic stem cells, comprising a protein comprising the same or substantially the same amino acid sequence as an amino acid sequence shown by SEQ ID NO:2 or 4.
[2] An agent for conferring hematopoiesis-supporting capacity to cells, comprising an expression vector harboring a nucleic acid that encodes a protein comprising the same or substantially the same amino acid sequence as an amino acid sequence shown by SEQ ID NO:2 or 4.
[3] An agent for maintaining or expanding hematopoietic stem cells, comprising a mammalian cell incorporating an expression vector harboring a nucleic acid that encodes a protein comprising the same or substantially the same amino acid sequence as an amino acid sequence shown by SEQ ID NO:2 or 4.
[4] The agent according to [3], wherein the mammalian cell is a stromal cell.
[5] The agent according to [3], wherein the mammalian cell does not possess hematopoiesis-supporting capacity.
[6] The agent according to any one of [3] to [5], wherein the expression vector is a lentivirus vector or a retrovirus vector.
[7] A method of producing a mammalian cell with improved hematopoiesis-supporting capacity, comprising introducing to a mammalian cell an expression vector harboring a nucleic acid that encodes a protein comprising the same or substantially the same amino acid sequence as an amino acid sequence shown by SEQ ID NO:2 or 4, and selecting a cell that expresses the nucleic acid.
[8] A method of maintaining or expanding hematopoietic stem cells, comprising culturing hematopoietic stem cells in the presence of a protein comprising the same or substantially the same amino acid sequence as an amino acid sequence shown by SEQ ID NO:2 or 4.
[9] The method according to [8], comprising culturing hematopoietic stem cells in the co-presence of at least one kind of cytokine.
[10] A method of maintaining or expanding hematopoietic stem cells, comprising co-culturing a mammalian cell incorporating an expression vector harboring a nucleic acid that encodes a protein comprising the same or substantially the same amino acid sequence as an amino acid sequence shown by SEQ ID NO:2 or 4, and a hematopoietic stem cell.
[11] The method according to [10], wherein the mammalian cell is a stromal cell.
[12] The method according to [10], wherein the mammalian cell does not possess hematopoiesis-supporting capacity.
[13] The method according to any one of [10] to [12], wherein the expression vector is a lentivirus vector or a retrovirus vector.
[14] A cell population containing expanded hematopoietic stem cells, obtained by the method according to any one of [8] to [13].
[15] An agent for ameliorating hematopoietic function, comprising the cell population according to [14] above.

### EFFECT OF THE INVENTION

Use of the method of the present invention makes it possible to efficiently maintain and expand hematopoietic stem cells ex vivo. A cell population containing hematopoietic stem cells obtained by the method can be applied to hematopoietic stem cell transplantation when administered to a living organism. Furthermore, because the agent of the present invention for maintenance/expansion of hematopoietic stem cells is capable of expanding hematopoietic stem cells in vivo or ex vivo, it can be used for maintenance/expansion of hematopoietic stem cells outside the body, and is also applicable to the treatment of a disease that hampers the normal genesis of blood cells when administered to a living organism.

### BRIEF DESCRIPTION OF THE DRAWINGS

FIG. 1 shows the effects of overexpression of candidate genes on the CFC frequency. A total of 80 CD34⁻KSL HSCs (10 cells/well) were co-cultured with PA6 cells, PA6 S-2 cells, or PA6 S-2 cells that express any of the indicated genes for 10 days, and then subjected to CFC assay. Each colony was scored according to the morphology thereof. The data shown indicate means for two separate experiments.

### BEST MODE FOR CARRYING OUT THE INVENTION

The present invention provides an agent for maintenance/expansion of hematopoietic stem cells comprising the HSC activity supporting factor of the present invention. Here, “a hematopoietic stem cell (HSC)” refers to a stem cell possessing both self-renewing capacity and multipotency for differentiation into all types of blood and lymphocyte-lineage cells; “maintenance” of hematopoietic stem cells refers to maintaining self-renewing capacity, the undifferentiated state and multipotency; “expansion” of hematopoietic stem cells refers to an increase in the number of cells possessing the above-described properties (capacities) by cell division.

The HSC activity supporting factor in the present invention is a protein comprising the same or substantially the same amino acid sequence as an amino acid sequence shown by SEQ ID NO: 2 or 4.

The HSC activity supporting factor in the present invention may be a protein isolated and purified from a cell (e.g., hepatocyte, splenocyte, nerve cell, glial cell, pancreatic β cell, myelocyte, mesangial cell, Langerhans' cell, epidermal cell, epithelial cell, goblet cell, endothelial cell, smooth muscle cell, fibroblast, fibrocyte, myocyte, adipocyte, immune cell (e.g., macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell, interstitial cell, or a corresponding precursor cell, stem cell or cancer cell thereof, and the like) of mammals (e.g., human, mouse, rat, monkey, chimpanzee, rabbit, sheep, pig, cow, horse, cat, dog and the like) or any tissue in which these cells are present [e.g., brain or any portion of brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gallbladder, bone marrow, adrenal gland, skin, lung, gastrointestinal tract (e.g., large intestine and small intestine), blood vessel, heart, thymus, spleen, submandibular gland, peripheral blood, prostate, testicle, ovary, placenta, uterus, bone, joint, adipose tissue (e.g., brown adipose tissue, white adipose tissue), skeletal muscle and the like] and the like. The HSC activity supporting factor may also be a protein biochemically synthesized in a chemical synthesis or cell-free translation system. Alternatively, the HSC activity supporting factor may be a recombinant protein produced by a transformant introduced with a nucleic acid having the base sequence that encodes the above-described amino acid sequence.

As “substantially the same amino acid sequence as an amino acid sequence shown by SEQ ID NO: 2 or 4”, an amino acid sequence having a homology of about 80% or more, preferably about 90% or more, more preferably about 95% or more, particularly preferably about 97% or more, and most preferably about 98% or more, with an amino acid sequence shown by SEQ ID NO: 2 or 4 can be mentioned. Here, the “homology” means a ratio (%) of identical amino acid residues and similar amino acid residues to all overlapping amino acid residues in the best alignment where two amino acid sequences are aligned using a mathematical algorithm known in the technical field (preferably, the algorithm considers introduction of gaps on one or both sides of the sequence for the best alignment). “A similar amino acid” means an amino acid having similar physiochemical properties; examples thereof include amino acids classified under the same group, such as aromatic amino acids (Phe, Trp, Tyr), aliphatic amino acids (Ala, Leu, Ile, Val), polar amino acids (Gln, Asn), basic amino acids (Lys, Arg, His), acidic amino acids (Glu, Asp), amino acids having a hydroxyl group (Ser, Thr) and amino acids having a small side-chain (Gly, Ala, Ser, Thr, Met). Substitution by such similar amino acids is expected to give no change in the phenotype of protein (i.e., conservative amino acid substitution). Specific examples of conservative amino acid substitution are obvious in the relevant technical field, and are described in various documents (see, for example, Bowie et al., Science, 247:1306-1310 (1990)).

Homology of the amino acid sequences in the present specification can be calculated under the following conditions (an expectation value=10; gaps are allowed; matrix=BLOSUM62; filtering=OFF) using a homology scoring algorithm NCBI BLAST (National Center for Biotechnology Information Basic Local Alignment Search Tool).

More preferably, the “substantially the same amino acid sequence as an amino acid sequence shown by SEQ ID NO: 2 or 4” is an amino acid sequence having an identity of about 80% or more, preferably about 90% or more, more preferably about 95% or more, particularly preferably about 97%, most preferably about 98% or more, with an amino acid sequence shown by SEQ ID NO: 2 or 4.

Alternatively, the “substantially the same amino acid sequence as an amino acid sequence shown by SEQ ID NO: 2 or 4” is (1) an amino acid sequence shown by SEQ ID NO: 2 or 4, wherein 1-30, preferably 1-20, more preferably 1-10, particularly preferably 1-2, 3, 4 or 5, amino acids have been deleted, (2) an amino acid sequence shown by SEQ ID NO: 2 or 4, wherein 1-30, preferably 1-20, more preferably 1-10, particularly preferably 1-2, 3, 4 or 5, amino acids have been added, (3) an amino acid sequence shown by SEQ ID NO: 2 or 4, wherein 1-30, preferably 1-20, more preferably 1-10, particularly preferably 1-2, 3, 4 or 5, amino acids have been inserted, (4) an amino acid sequence shown by SEQ ID NO: 2 or 4, wherein 1-30, preferably 1-20, more preferably 1-10, particularly preferably 1-2, 3, 4 or 5, amino acids have been substituted by other amino acids, or (5) an amino acid sequence which is a combination thereof.

When the amino acid sequence is inserted, deleted or substituted as mentioned above, the insertion, deletion or substitution site thereof is not particularly limited as long as the activity of the protein is retained.

Preferable examples of the “substantially the same amino acid sequence as an amino acid sequence shown by SEQ ID NO: 2 or 4” include the amino acid sequences shown by SEQ ID NOs: 20 and 22.

“A protein comprising substantially the same amino acid sequence as an amino acid sequence shown by SEQ ID NO:2 or 4” means a protein containing the above-described “substantially the same amino acid sequence as an amino acid sequence shown by SEQ ID NO:2 or 4”, and possessing substantially the same quality of activity as a protein comprising an amino acid sequence shown by SEQ ID NO:2 or 4.

Here, “substantially the same quality of activity” means the activity of supporting the self-renewing capacity, undifferentiated state, and potency for differentiation into all blood and lymphocyte-lineage cells (these are also generically referred to as “HSC activity”) of hematopoietic stem cells. “Substantially the same quality” means that the activities are qualitatively equivalent to each other. Therefore, it is preferable that the HSC activity supporting activities be equivalent to each other, but the quantitative factors of these activities, such as the extent of activity (e.g., about 0.5 to about 2 folds) and the molecular weight of the protein, may be different.

HSC activity supporting activity (hematopoiesis supporting capacity) can be determined by a method known per se (e.g., J Exp Med 192 (2000) 1281-1288, details are given in Examples below).

Alternatively, “a protein comprising substantially the same amino acid sequence as an amino acid sequence shown by SEQ ID NO:2 or 4” is an orthologue of the human TMEM140 protein, which consists of the amino acid sequence shown by SEQ ID NO:2 (GenBank accession number: NP_{—}060765), or the mouse TMEM140 protein, which consists of the amino acid sequence shown by SEQ ID NO:4 (GenBank accession number: NP_{—}932103), in another mammal (e.g., rat and chimpanzee orthologues registered with GenBank under accession numbers NP_{—}001009709 and XP_{—}001143821, respectively, and the like).

In the present specification, the left end of the proteins and peptides indicates the N-terminus (amino terminus) and the right end thereof indicates the C-terminus (carboxyl terminus), according to the common practice of peptide designation. For HSC activity supporting factor of the present invention, including a protein having an amino acid sequence shown by SEQ ID NO: 2 or 4, the C-terminus may be any of a carboxyl group (—COOH), a carboxylate (—COO⁻), an amide (—CONH₂) or an ester (—COOR). Here, as R in the ester, a C₁₋₆ alkyl group such as methyl, ethyl, n-propyl, isopropyl and n-butyl, a C₃₋₈ cycloalkyl group such as cyclopentyl and cyclohexyl, a C₆₋₁₂ aryl group such as phenyl and α-naphthyl, a phenyl-C₁₋₂ alkyl group such as benzyl and phenethyl, a C₇₋₁₄ aralkyl group such as an α-naphthyl-C₁₋₂ alkyl group such as α-naphthylmethyl, a pivaloyloxymethyl group; and the like can be used.

When the HSC activity supporting factor of the present invention has a carboxyl group (or a carboxylate) in addition to that on the C-terminal, one in which the carboxyl group is amidated or esterified is also included in the protein in the present invention. In this case, as the ester, the above-described C-terminal ester and the like, for example, can be used.

Furthermore, the HSC activity supporting factor of the present invention also includes a protein wherein the amino group of the N-terminal amino acid residue thereof is protected by a protecting group (e.g., a C₁₋₆ acyl group such as C₁₋₆ alkanoyl such as a formyl group or an acetyl group, and the like), a protein wherein the N-terminal glutamine residue, which is produced by cleavage in vivo, has been converted to pyroglutamic acid, a protein wherein a substituent (e.g., —OH, —SH, an amino group, an imidazole group, an indole group, a guanidino group and the like) on an amino acid side chain in the molecule is protected by an appropriate protecting group (e.g., a C₁₋₆ acyl group such as a C₁₋₆ alkanoyl group such as a formyl group or an acetyl group, and the like), a conjugated protein such as what is called a glycoprotein, which has a sugar chain bound thereto, and the like.

The HSC activity supporting factor of the present invention may be in a free form or a salt. Examples of the salts of the HSC activity supporting factor of the present invention include physiologically acceptable salts with acids or bases, and physiologically acceptable acid addition salts are particularly preferable. Such salts include, for example, salts with inorganic acids (e.g., hydrochloric acid, phosphoric acid, hydrobromic acid, sulfuric acid), or salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

The HSC activity supporting factor of the present invention can also be produced from the cells or tissues of the above-described mammals by a method of protein purification known per se. Specifically, the HSC activity supporting factor can be produced by homogenizing a tissue or cells of a mammal, removing cell debris by low speed centrifugation, precipitating cell membrane-containing fractions by high speed centrifugation of the supernatant (and, where necessary, purifying the cell membrane fractions by density gradient centrifugation etc.), and subjecting the fraction to chromatographies such as reversed-phase chromatography, ion exchange chromatography, affinity chromatography and the like.

Alternatively, the HSC activity supporting factor of the present invention can also be produced according to known peptide synthesis method.

The method of peptide synthesis may be any of, for example, a solid phase synthesis process and a liquid phase synthesis process. That is, a desired protein can be produced by condensing a partial peptide or amino acids capable of constituting the HSC activity supporting factor and the remaining portion, and eliminating any protecting group the resultant product may have.

As examples of the commonly known methods of condensation and elimination of the protecting group, the methods described in (1) to (5) below can be mentioned.

### (1) M. Bodanszky and M. A. Ondetti, Peptide Synthesis, Interscience Publishers, New York (1966)

### (2) Schroeder and Luebke, The Peptide, Academic Press, New York (1965)

(3) Nobuo Izumiya, et al.: Peptide Gosei-no-Kiso to Jikken, published by Maruzen Co. (1975);

### (4) Haruaki Yajima and Shunpei Sakakibara: Seikagaku Jikken Koza 1, Tanpakushitsu no Kagaku IV, 205 (1977)

(5) Haruaki Yajima, ed.: Zoku Iyakuhin no Kaihatsu, Vol. 14, Peptide Synthesis, published by Hirokawa Shoten.

The protein thus obtained can be isolated and purified by a known purification method. Examples of the purification method include solvent extraction, distillation, column chromatography, liquid chromatography, recrystallization and a combination of these.

When the protein obtained by the above-described method is a free form, the free form can be converted to an appropriate salt by a publicly known method or a method based thereon; conversely, when the protein is obtained in the form of a salt, the salt can be converted to a free form or another salt by a publicly known method or a method based thereon.

Moreover, the HSC activity supporting factor of the present invention can also be produced by culturing a transformant having the nucleic acid encoding same, and separating and purifying the HSC activity supporting factor of the present invention from the obtained culture. The nucleic acid that encodes the HSC activity supporting factor of the present invention may be DNA or RNA, or a DNA/RNA chimera, and is preferably DNA. In addition, the nucleic acid may be a double-strand, or single-strand. The double-strand may be a double-stranded DNA, a double-stranded RNA, or a DNA:RNA hybrid. In the case of a single strand, it may be a sense strand (i.e., coding strand) or an antisense strand (i.e., non-coding strand).

As the DNA encoding the HSC activity supporting factor of the present invention, genomic DNA, genomic DNA library, cDNA derived from any cell [for example, hepatocyte, splenocyte, nerve cell, glial cell, pancreatic β cells, myeloid cell, mesangial cell, Langerhans' cell, epidermal cell, epithelial cell, goblet cell, endothelial cell, smooth muscle cell, fibroblast, fibrocyte, myocytes, adipocyte, immune cell (e.g., macrophage, T cell, B cell, natural killer cell, mast cell, neutrophil, basophil, eosinophils, monocyte), megakaryocyte, synovial cell, chondrocytes, bone cell, osteoblast, osteoclast, mammary cell, hepatocyte or interstitial cell, or a corresponding precursor cell, stem cell, cancer cell and the like] of mammal (e.g., human, cow, monkey, horse, pig, sheep, goat, dog, cat, guinea pig, rat, mouse, rabbit, hamster and the like), or any tissue where such cells are present [e.g., brain or any portion of brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gallbladder, bone marrow, adrenal gland, skin, lung, gastrointestinal tract (e.g., large intestine, small intestine), blood vessel, heart, thymus, spleen, submandibular gland, peripheral blood, prostate, testicle, ovary, placenta, uterus, bone, joint, adipose tissue (e.g., brown adipose tissue, white adipose tissue), skeletal muscle, and the like], synthetic DNA and the like can be mentioned. A genomic DNA and cDNA encoding the HSC activity supporting factor of the present invention can also be directly amplified by Polymerase Chain Reaction (hereinafter to be abbreviated as “PCR method”) or Reverse Transcriptase-PCR (hereinafter to be abbreviated as “RT-PCR method”), using genomic DNA fraction or total RNA or mRNA fraction prepared from the above-mentioned cell/tissue as a template. Alternatively, genomic DNA or cDNA encoding the HSC activity supporting factor of the present invention can also be cloned by colony or plaque hybridization method, PCR method and the like, from the genomic DNA library or cDNA library prepared by inserting, into a suitable vector, a fragment of genomic DNA and total RNA or mRNA prepared from the above-mentioned cell/tissue. The vector to be used for the library may be any of bacteriophage, plasmid, cosmid, phagemid and the like.

A DNA encoding the HSC activity supporting factor of the present invention is not particularly limited as long as it is a DNA encoding the above-mentioned “protein containing an amino acid sequence substantially the same as an amino acid sequence shown by SEQ ID NO: 2 or 4”. Preferably, it is a DNA that contains a base sequence shown by SEQ ID NO: 1 or 3, a DNA that contains a base sequence hybridizing to a base sequence shown by SEQ ID NO: 1 or 3 under stringent conditions and encodes a protein having substantially the same quality of activity as the aforementioned protein comprising an amino acid sequence shown by SEQ ID NO: 2 or 4 (that is, HSC activity supporting activity (hematopoiesis supporting ability)).

Useful “DNA capable of hybridizing with the base sequence shown by SEQ ID NO:1 or 3 under stringent conditions” include, for example, a DNA comprising a base sequence having a homology of about 80% or more, preferably about 90% or more, more preferably about 95% or more, particularly preferably about 97% or more, most preferably about 98% or more, to the base sequence shown by SEQ ID NO:1 or 3. Homology of the base sequences in the present specification can be calculated under the following conditions (an expectation value=10; gaps are allowed; filtering=ON; match score=1; mismatch score=−3) using a homology scoring algorithm NCBI BLAST (National Center for Biotechnology Information Basic Local Alignment Search Tool).

The hybridization can be performed by a method known per se or a method analogous thereto, for example, a method described in Molecular Cloning, 2nd ed. (J. Sambrook et al., Cold Spring Harbor Lab. Press, 1989) and the like. A commercially available library can also be used according to the instructions of the attached manufacturer's protocol. The stringent conditions refer to, for example, conditions involving a sodium salt concentration of about 19 to about 40 mM, preferably about 19 to about 20 mM, and a temperature of about 50 to about 70° C., preferably about 60 to about 65° C. In particular, a case wherein the sodium concentration is about 19 mM and the temperature is about 65° C. is preferred.

Preferable examples of the DNA encoding the HSC activity supporting factor of the present invention include DNAs comprising the base sequence shown by SEQ ID NO: 19 or 21.

Alternatively, the DNA that encodes the HSC activity supporting factor of the present invention is an orthologue of a DNA comprising the base sequence that encodes the human TMEM140 protein shown by SEQ ID NO:1 (GenBank accession number: NM_{—}018295) or a DNA comprising the base sequence that encodes the mouse TMEM140 protein shown by SEQ ID NO:3 (GenBank accession number: NM_{—}197986) in another mammal (e.g., rat and chimpanzee orthologues registered with GenBank under accession numbers NM_{—}001009709 and XM_{—}001143821, respectively, and the like).

The DNA that encodes the HSC activity supporting factor of the present invention can be cloned by amplifying it by the PCR method using a synthetic DNA primer having a portion of the base sequence that encodes the protein, or by hybridizing DNA incorporated in an appropriate expression vector to a labeled DNA fragment or synthetic DNA that encodes a portion or the entire region of the protein. The hybridization can be performed by, for example, a method described in Molecular Cloning, 2nd ed. (ibid.) and the like. A commercially available library can also be used according to the instructions of the manufacturer's protocol attached thereto.

The base sequence of the DNA can be converted according to a method known per se, such as the ODA-LA PCR method, the Gapped duplex method, or the Kunkel method, or a method based thereon, using a commonly known kit, for example, Mutan™-super Express Km (TAKARA SHUZO CO. LTD.), Mutan™-K (TAKARA SHUZO CO. LTD.) and the like.

The cloned DNA can be used as is, or after digestion with a restriction enzyme or addition of a linker as desired, depending on the purpose of its use. The DNA may have the translation initiation codon ATG at the 5′ end thereof, and the translation stop codon TAA, TGA or TAG at the 3′ end thereof. These translation initiation codons and translation stop codons can be added by using a suitable synthetic DNA adaptor.

An expression vector containing a DNA encoding the HSC activity supporting factor of the present invention can be produced, for example, by cleaving out an object DNA fragment from the DNA and connecting the DNA fragment at the downstream of a promoter in a suitable expression vector.

Useful expression vectors include plasmids derived from E. coli (e.g., pBR322, pBR325, pUC12, pUC13); plasmids derived from Bacillus subtilis (e.g., pUB110, pTP5, pC194); plasmids derived from yeast (e.g., pSH19, pSH15); insect cell expression plasmid (e.g., pFast-Bac); animal cell expression plasmid (e.g., pA1-11, pXT1, pRc/CMV, pRc/RSV, pcDNAI/Neo); bacteriophages such as λ phage; insect virus (e.g., baculovirus and the like) vector (e.g., BmNPV, AcNPV); animal virus (e.g., lentivirus, adenovirus, retrovirus, adeno-associated virus, herpesvirus, vaccinia virus, pox virus, polio virus and the like) vector and the like.

The promoter may be any promoter appropriate for the host used to express the gene. For example, when an animal cell is used as the host, the SRα promoter, the SV40 promoter, the LTR promoter, the CMV (cytomegalovirus) promoter, RSV (Rous sarcoma virus) promoter, MoMuLV (Moloney murine leukemia virus) LTR, HSV-TK (herpes simplex virus thymidine kinase) promoter, EF-1α (elongation factor) promoter, UbC (ubiquitin C) promoter and the like can be mentioned.

Useful expression vectors include, in addition to the above, expression vectors that optionally comprise an enhancer, a splicing signal, a polyA addition signal, a selection marker gene, a reporter gene, an SV40 replication origin, and the like. As examples of the selection marker gene, drug resistance gene such as the dihydrofolate reductase gene (dhfr, methotrexate (MTX) resistance), the ampicillin resistance gene, the neomycin resistance gene (G418 resistance), and the like, and a gene complementing auxotrophic mutation and the like can be mentioned. As the reporter gene, a gene encoding luciferase, green fluorescence protein (GFP), Venus and the like can be mentioned.

An HSC activity supporting factor can be produced by transforming a host with an expression vector containing a DNA encoding the above-mentioned HSC activity supporting factor and culturing the resulting transformant. As useful examples of the host, a bacterium of the genus Escherichia, a bacterium of the genus Bacillus, yeast, an insect cell, an insect, an animal cell, and the like can be mentioned. Transformation can be performed according to the choice of host by a commonly known method.

Cultivation of the transformant can be performed according to the choice of host by a commonly known method.

Useful medium for cultivating a transformant whose host is an animal cell include, for example, minimum essential medium (MEM), Dulbecco's modified Eagle's Medium (DMEM), RPMI 1640 medium, 199 medium and the like, which are supplemented with about 5 to about 20% fetal bovine serum. The medium's pH is preferably about 6 to about 8. Cultivation is normally performed at about 30 to about 40° C. for about 15 to about 60 hours, and the culture may be aerated or agitated as necessary.

Thus, the HSC activity supporting factor of the present invention can be produced in or outside the cells of the transformant.

The HSC activity supporting factor of the present invention can be separated and purified according to a method known per se from the culture obtained by cultivating the aforementioned transformant.

For example, a method is used as appropriate wherein the bacteria or cells are collected from the culture by a known means, suspended in an appropriate buffer solution, and disrupted by means of sonication, lysozyme and/or freeze-thawing and the like, after which cell debris is removed by low speed centrifugation, cell membrane-containing fractions are precipitated by high speed centrifugation of the supernatant (and, where necessary, the cell membrane fractions are purified by density gradient centrifugation etc.), and the like. Isolation and purification of the HSC activity supporting factor of the present invention contained in the thus-obtained membrane fraction can be conducted according to a method know per se. Useful methods include methods based on solubility, such as salting-out and solvent precipitation; methods based mainly on differences in molecular weight, such as dialysis, ultrafiltration, gel filtration, and SDS-polyacrylamide gel electrophoresis; methods based on differences in electric charge, such as ion exchange chromatography; methods based on specific affinity, such as affinity chromatography; methods based on differences in hydrophobicity, such as reverse phase high performance liquid chromatography; methods based on differences in isoelectric point, such as isoelectric focusing; and the like. These methods can be combined as appropriate.

Furthermore, the HSC activity supporting factor of the present invention can also be synthesized in vitro using a cell-free protein translation system that comprises a rabbit reticulocyte lysate, wheat germ lysate, Escherichia coli lysate and the like, with RNA corresponding to the above-described DNA that encodes the protein as the template. Alternatively, it can be synthesized using a cell-free transcription/translation system containing RNA polymerase, with the DNA that encodes the HSC activity supporting factor of the present invention as the template.

In the agent of the present invention for maintenance/expansion of hematopoietic stem cells, the HSC activity supporting factor of the present invention, obtained as described above, can be used as the above-described drug as it is, or after being mixed with a cytophysiologically acceptable carrier to obtain a composition as required. For example, the agent of the present invention for maintenance/expansion of hematopoietic stem cells can be prepared by dissolving the HSC activity supporting factor of the present invention in water or an appropriate buffer solution (e.g., phosphate buffer solution, PBS, tris-HCl buffer solution and the like) to obtain an appropriate concentration. A preservative, stabilizer, reducing agent, isotonizing agent and the like in common use may be formulated as required.

The agent of the present invention for maintenance/expansion of hematopoietic stem cells can be used to maintain/expand hematopoietic stem cells by, for example, adding an effective amount of the HSC activity supporting factor of the present invention to the medium, and culturing hematopoietic stem cells. Accordingly, the present invention also provides a method of maintaining/expanding hematopoietic stem cells, comprising culturing hematopoietic stem cells in the presence of the HSC activity supporting factor of the present invention.

Hematopoietic stem cells can be collected from bone marrow, fetal livers, umbilical cord blood, peripheral blood and the like of mammals such as humans and mice by a method known per se (e.g., Science 273 (1996) 242-245; described in an Example below). The hematopoietic stem cells used in the present invention may be stem cells alone, or a homogenous cell population comprising stem cells at high frequency.

Examples of media used to culture hematopoietic stem cells include a minimum essential medium (MEM) containing about 5 to 20% bovine fetal serum, Dulbecco's modified Eagle medium (DMEM), RPMI 1640 medium, 199 medium and the like. As required, cytokines such as stem cell factor (SCF), interleukin-3 (IL-3), interleukin-6 (IL-6), interleukin-7 (IL-7), interleukin-11 (IL-11), fms-like tyrosine kinase-3 (Flt-3) ligand (FLT), erythropoietin (EPO), and thrombopoietin (TPO), hormones such as insulin, transportation proteins such as transferrin, and the like may further be contained in the medium. Particularly, cytokines are preferably contained, and the number of cytokines contained in the medium may be 1 or 2 or more. SCF, in particular, is essential. The concentration of a cytokine added to the medium is normally 1 to 500 ng/mL, preferably 5 to 300 ng/mL, more preferably 10 to 100 ng/mL. The pH of the medium is preferably about 6 to about 8.

The agent of the present invention for maintenance/expansion of hematopoietic stem cells can be added to the above-described medium to obtain a concentration of the HSC activity supporting factor of the present invention of 1 to 500 ng/mL, preferably 5 to 300 ng/mL, more preferably 10 to 100 ng/mL. The hematopoietic stem cells can be added to the above-described medium to obtain a cell density in common use in the art. Cultivation is normally performed at about 30° C. to about 40° C. in an atmosphere of about 5 to about 10% CO₂ for a time sufficient to achieve the desired expansion. The culture may be aerated or agitated as necessary.

In another preferred embodiment of the present invention, mammalian cells incorporating an expression vector harboring a nucleic acid that encodes the HSC activity supporting factor of the present invention and hematopoietic stem cells are co-cultured, whereby maintenance/expansion of hematopoietic stem cells can be achieved. Accordingly, the present invention also provides (1) an agent that confers hematopoiesis-supporting capacity to cells containing an expression vector harboring a nucleic acid that encodes the HSC activity supporting factor of the present invention, (2) a method of producing mammalian cells with improved hematopoiesis-supporting capacity, comprising introducing the expression vector to mammalian cells, and selecting cells that express the nucleic acid, and (3) an agent for maintenance/expansion of hematopoietic stem cells, comprising mammalian cells incorporating the expression vector.

As “an expression vector harboring a nucleic acid that encodes the HSC activity supporting factor of the present invention”, the above-described expression vector that can be used for recombination production of the HSC activity supporting factor of the present invention can likewise be used preferably. As the background vector for the expression vector, viral vectors such as lentivirus, adenovirus, retrovirus, adeno-associated virus, herpesvirus, vaccinia virus, poxvirus, and poliovirus can be mentioned; retrovirus and lentivirus vectors are preferred, with greater preference given to lentivirus vector.

The host mammalian cell into which the expression vector is introduced is not particularly limited, but is preferably a stromal cell. Stromal cells can be isolated from bone marrow, bone, fetal liver, aorta-gonad-mesonephros region and the like of a mammal by a method known per se.

Some stromal cells are known to be capable of supporting maintenance/expansion of hematopoietic stem cells by nature, such as OP9 cells, used in an Example below, and PA6 cells; the stromal cells used in the present invention may have hematopoiesis supporting capacity per se or not. Hence, the hematopoiesis supporting capacity conferring agent of the present invention not only confers hematopoiesis supporting capacity to cells that do not possess hematopoiesis supporting capacity, but also is effective in further improving the hematopoiesis supporting capacity, to cells that possess the capacity by nature. However, because it is uneasy to isolate and acquire cells that possess excellent hematopoiesis supporting capacity per se, the hematopoiesis supporting capacity conferring agent of the present invention is of paramount significance in that it is capable of conferring hematopoiesis supporting capacity to the cells when introduced into stromal cells that do not possess hematopoiesis supporting capacity by nature.

Introduction of an expression vector to a mammalian cell can be achieved by a technique known per se. For example, when a lentivirus vector is used, the method described in Exp. Hematol., 30 (2002), 11-17 and the like can be used. Although the expression of the introduced nucleic acid that encodes the HSC activity supporting factor of the present invention can also be selected, for example, with drug resistance as an index using a selection marker gene inserted into the expression vector, gene transfer efficiency can be measured microscopically, without exerting a selection pressure, provided that a visualizable reporter gene is inserted into the expression vector in advance.

Mammalian cells, preferably stromal cells, incorporating an expression vector harboring a nucleic acid that encodes the HSC activity supporting factor of the present invention, obtained as described above, can be provided as an agent for maintenance/expansion of hematopoietic stem cells, for example, in suspension in an appropriate maintenance medium as described above.

The agent for maintenance/expansion of hematopoietic stem cells can be used to maintain/expand hematopoietic stem cells by, for example, adding to the medium mammalian cells in an amount sufficient to produce an effective amount of the HSC activity supporting factor of the present invention, and co-culturing the cells with hematopoietic stem cells.

The hematopoietic stem cells used, the medium used to culture the cells, the cytokines, hormones, and other proteins added to the medium, and the like may be the same as those described above.

The agent of the present invention for maintenance/expansion of hematopoietic stem cells can be added to the above-described medium to obtain a cell density in common use for a feeder cell in the art. The hematopoietic stem cells can be added to the above-described medium to obtain a cell density in common use in the art. Cultivation is normally performed at about 30° C. to about 40° C. in an atmosphere of about 5 to about 10% CO₂ for a time sufficient to achieve the desired expansion. The culture may be aerated or agitated as necessary.

The present invention also provides a cell population containing expanded hematopoietic stem cells, obtained by the above-described method, and a hematopoietic function ameliorating agent in a mammal, comprising the cell population containing hematopoietic stem cells. The hematopoietic function ameliorating agent of the present invention can be used preferably as a prophylactic and/or therapeutic agent for diseases accompanied by a disturbance of hematopoietic function, for example, aplastic anemia, congenital immunodeficiencies, inborn errors of metabolism, osteomyelodysplasia, leukemia, malignant lymphoma, multiple myeloma, myelofibrosis and the like.

Here, “a cell population containing hematopoietic stem cells” means a cell population comprising expanded hematopoietic stem cells, obtained by the method of expansion of the present invention, and it is not always necessary to isolate and purify the expanded hematopoietic stem cells only. It is generally known that when hematopoietic stem cells are allowed to expand themselves ex vivo, it is impossible to selectively expand hematopoietic stem cells only, and that the cell population obtained will contain blood and lymphocyte-lineage cells at all differentiation stages. However, these cells are also required for a living organism; administration of the entire expanded cell population to the living organism is expected to improve hematopoietic function. In particular, in cell transplantation intended to treat a mammal with impaired hematopoietic function, quick amelioration of the hematopoietic function is demanded; a better therapeutic effect is expected by transplanting a heterogeneous cell population comprising multiple lineages of blood cells that have differentiated to some degree, rather than by transplanting homogenous undifferentiated hematopoietic stem cells. Of course, a homogenous hematopoietic stem cell population comprising nothing more than hematopoietic stem cells maintaining the undifferentiated state are also included in “a cell population containing hematopoietic stem cells”. Such a homogenous cell population can be acquired from the above-described heterogeneous cell population by using a technique known per se based on FACS or the like.

The hematopoietic function ameliorating agent of the present invention can be used after the above-described cell population containing hematopoietic stem cells is mixed with a pharmacologically acceptable carrier to obtain a pharmaceutical composition as required.

Here, as the pharmacologically acceptable carrier, various organic or inorganic carrier substances in common use as materials for formulating pharmaceuticals are used; for example, these are formulated as suspending agents in suspensions, isotonizing agents, buffering agents, soothing agents and the like. Pharmaceutical additives such as antiseptics, antioxidants, thickeners, and stabilizers can also be used as required.

Because of its low toxicity, the preparation thus obtained can be safely administered to mammals (e.g., humans, monkeys, dogs, cats, mice, rats, rabbits, sheep, pigs and the like), preferably to humans. It is desirable that a cell population containing hematopoietic stem cells derived from the same animal species as the mammal being the subject of administration be used, and it is particularly desirable that a cell population containing hematopoietic stem cells derived from hematopoietic stem cells collected from an individual mammal being the subject of administration be used.

Although the hematopoietic function ameliorating agent of the present invention may be administered by any route, parenteral administration (e.g., intravenous injection, topical injection and the like) is preferred. Suitable preparations include aqueous and non-aqueous isotonic sterile injectable liquids.

Although the dosage of the hematopoietic function ameliorating agent of the present invention varies depending on the activity and kind of the active ingredient, seriousness of illness, recipient animal species, the recipient's drug tolerance, body weight, age, and the like, the amount of hematopoietic stem cells per dose for an adult is normally 10⁶ cells/kg or more, preferably 10⁶ cells/kg to 10¹⁰ cells/kg, more preferably 2×10⁶ cells/kg to 10⁹ cells/kg.

The agent of the present invention for maintenance/expansion of hematopoietic stem cells, which comprises the HSC activity supporting factor of the present invention, can promote maintenance/expansion of hematopoietic stem cells in the body of a mammal, when the protein is administered to the animal as it is, or after being mixed with a pharmacologically acceptable carrier to obtain a pharmaceutical composition as required. Therefore, the agent of the present invention for maintenance/expansion of hematopoietic stem cells can be used preferably as a prophylactic and/or therapeutic agent for diseases accompanied by a disturbance of hematopoietic function, for example, aplastic anemia, congenital immunodeficiencies, inborn errors of metabolism, osteomyelodysplasia, leukemia, malignant lymphoma, multiple myeloma, myelofibrosis and the like.

Examples of the pharmacologically acceptable carrier include, but are not limited to, excipients such as sucrose, starch, glucose and cellulose; binders such as gelatin, acacia and polyethylene glycol; disintegrants such as starch, carboxymethylcellulose, hydroxypropyl starch and sodium-glycol-starch; lubricants such as magnesium stearate, Aerosil, talc and sodium lauryl sulfate; flavoring agents such as citric acid, menthol and glycyrrhizin ammonium salt; preservatives such as sodium benzoate, sodium hydrogen sulfite, methyl paraben and propyl paraben; stabilizers such as citric acid, sodium citrate and acetic acid; suspending agents such as methylcellulose, polyvinylpyrrolidone and aluminum stearate; dispersing agents such as surfactants; diluents such as water, physiological saline and orange juice; base waxes such as cacao butter, polyethylene glycol and refined kerosene; and the like.

Preparations suitable for oral administration are a liquid comprising an effective amount of the material dissolved in a diluent like water, physiological saline or orange juice, a capsule, sachet, tablet, suspension, emulsion, and the like.

As preparations suitable for parenteral administration (e.g., subcutaneous injection, intramuscular injection, topical injection, intraperitoneal administration and the like), aqueous and non-aqueous isotonic sterile injectable liquids are available, which may contain a suspending agent, a solubilizer, a thickener, a stabilizer, an antiseptic and the like. The preparation, like ampoules and vials, can be enclosed in a container for a unit dosage or a multiple dosage. Also, an active ingredient and a pharmaceutically acceptable carrier can also be freeze-dried and stored in a state that only requires dissolution or suspending in an appropriate sterile vehicle immediately before use.

Although the dosage of the agent of the present invention for maintenance/expansion of hematopoietic stem cells varies depending on the activity and kind of the active ingredient, seriousness of illness, recipient animal species, the recipient's drug tolerance, body weight, age, and the like, the amount of the active ingredient (HSC activity supporting factor of the present invention) per dose for an adult is normally 0.0001 to 500 mg/kg, preferably 0.0005 to 50 mg/kg, more preferably 0.001 to 5 mg/kg.

### EXAMPLES

The present invention is hereinafter described in more detail by means of the following Examples, to which, however, the invention is never limited.

### Example 1

### 1. Breeding of Mice

C57BL/6 (B6-Ly5.2) mice were purchased from Charles River Laboratories Japan Inc. (Yokohama, Japan). C57BL/6 mice congenic for the Ly5 locus (B6-Ly5.1) were obtained from the RIKEN BioResource Center (BRC). B6-Ly5.1/Ly5.2 F1 mice were acquired by mating pairs of B6-Ly5.1 and B6-Ly5.2 mice.

### 2. Cultivation of Stromal Cells and Transduction Using Lentivirus

An OP9 stromal cell line was obtained from the RIKEN BRC Cell Bank. PA6 and OP9 cells were maintained in an MEM-A (Sigma-Aldrich) containing 20% fetal bovine serum (FBS) (Sigma-Aldrich) at 37° C. in an environment of 5% CO₂. FANTOM cDNA clones corresponding to the genes used in this study were subcloned into the lentivirus vector plasmid pCSII-EF-MCS-IRES2-Venus. A recombinant lentivirus vector was prepared by the method described in Exp. Hematol. 30 (2002), 11-17. PA6 subclone cells were transduced using a lentivirus vector expressing a cDNA at a multiplicity of infection of 200, and a transfection efficiency of >90% was confirmed by fluorescence-activated cell sorting (FACS) analysis of Venus expression.

### 3. Purification of CD34⁻KSL Cells and Co-Culture with Stromal Cells

CD34^{-/low}c-Kit⁺Sca-1⁺ differentiation-specific antigen marker (lineage marker)⁻ (CD34⁻KSL) cells were purified by the method described in Science 273 (1996), 242-245, with a slight modification. Specifically, myelocytes isolated from B6-Ly5.2 mice at 10 to 16 weeks of age were stained with a differentiation-specific antigen marker antibody mixture consisting of biotinylated anti-Gr-1, anti-Mac-1, anti-B220, anti-IgM, anti-CD4, anti-CD8, and anti-Ter119 antibodies (eBioscience (San Diego, Calif.)). Differentiation-specific antigen marker⁺ cells were removed using streptavidin-coupled Dynabeads M-280 (Invitrogen (Carlsbad, Calif.)). The remaining cells were stained with fluorescein isothiocyanate (FITC)-conjugated anti-CD34, phycoerythrin (PE)-conjugated anti-Sca-1, and allophycocyanine (APC)-conjugated anti-c-Kit antibodies (all obtained from BD Biosciences (San Jose, Calif.)). The biotinylated antibodies were detected using streptavidin-APC-Cy7 (BD Biosciences). FACS was performed using FACSVantage SE (BD Biosciences). CD34⁻KSL cells were sorted into the individual wells of a 96-well plate containing stromal cells exposed to 1.5 Gy radiation (1×10⁴ cells/well), and co-cultured in 150 μL of an MEM-α containing 20% FBS.

### 4. Colony Forming Cell (CFC) Assay

After co-culture for 10 days, all cells in the wells were collected, and sown to a 12-well plate containing 0.6 mL of a methylcellulose medium (MethoCult GF M3434) (StemCell Technologies) containing 50 ng/mL rmSCF, 10 ng/mL rmIL-3, 10 ng/mL rhIL-6, and 3 units/mL rhEPO. After incubation for 12 days, colonies were recovered, cyto-spun onto a glass slide, and then stained with Hemacolor (Merck KGaA (Darmstadt, Germany)) for morphological examination. Colony forming unit-granulocytes-erythrocytes-monocytes-megakaryocytes (CFU-GEMM), CFU-granulocytes-erythrocytes-monocytes (CFU-GEM), CFU-granulocytes-monocytes (CFU-GM), CFU-granulocytes (CFU-G), CFU-monocytes (CFU-M), and burst forming unit-erythrocytes (BFU-E) were scored using standard scoring criteria. Total colony counts were taken in terms of CFC unit.

### 5. Competitive Bone Marrow Repopulation Assay

As explained in J Exp Med 192 (2000), 1281-1288, competitive bone marrow repopulation assay was performed using a congenic Ly5 mouse system. After co-culture, all cells in the wells were mixed with a total of 2×10⁵ bone marrow competitor cells from a B6-Ly5.1 mouse, and transplanted to a B6-Ly5.1 mouse (recipient mouse), previously exposed to a lethal dose (9.5 Gy) of radiation. After 12 to 16 weeks of transplantation, peripheral blood was collected from the recipient mouse by retrobulbar bleeding. After erythrocytes were lysed with ammonium chloride buffer, the remaining nucleated cells were stained with FITC-conjugated anti-Ly5.2, PE-conjugated anti-Ly5.1, biotinylated anti-Mac1, and biotinylated anti-Gr1 antibodies, and then added. At the same time, the cells were stained with APC-conjugated anti-B220 antibody or a mixture of APC-conjugated anti-CD4 and anti-CD8 antibodies. FACS analysis was performed using FACSCalibur. Donor chimerism was determined as a ratio of Ly5.2⁺ cells. If the ratio of chimerism in all myeloid systems, B lymphoid, and T lymphoid was >1.0%, it was judged that the hematopoiesis in the recipient mouse was reconstituted by the donor cells.

### 6. Microarray Analysis

Total RNA was isolated from FACS-sorted cells by using the ISOGEN reagent (Nippon Gene (Japan)). A biotin-labeled cRNA was prepared using 1 μg of total RNA, a 1-cycle cDNA synthesis kit and a 3′ amplification reagent for IVT labeling (Affymetrix (Santa Clara, Calif.)), and hybridized to the Affymetrix GeneChip Mouse Genome 430 2.0 array (Affymetrix), which comprised approximately 45,000 probe sets for analysis of the expression levels of more than 34,000 mouse genes. After washing and staining by an antibody amplification technique, and the microarray was scanned with Affymetrix GeneChip Scanner 3000 7G. All these techniques were performed according to the instructions of the manufacturer's protocol. The expression value (Signal) and detected level (present (P), absent (A), or marginal (M)) for each probe set were calculated using GeneChip Operating Software version 1.4 (Affymetrix). Signal values were standardized to obtain a mean of 100 in each experiment, whereby the slight differences thereamong were adjusted. For each probe set, a change value (Signal logarithmic ratio) and a change signal (Increase, Marginal Increase, No Change, Marginal Decrease, or Decrease) were calculated using the Comparison Analysis software program. All experiments were performed in duplicate. To identify differentially expressed genes, we selected probe sets exhibiting both a change signal of Increase and a Signal logarithmic ratio value of ≧1 (more than 2-fold upregulation), or both a change signal of Decrease and a Signal logarithmic ratio value of ≦−1 (more than 2-fold down regulation), in each of two separate experiments.

### 7. Quantitative Real Time Polymerase Chain Reaction (PCR)

Total RNA was isolated from 1×10⁶ stromal cells by using the ISOGEN reagent, and cDNA was synthesized from 1 μg of the total RNA by using SuperScript II reverse transcriptase (Invitrogen). Two separate cell samples were subjected to real time PCR amplification according to the instructions of the manufacturer's protocol using the LightCycler FastStart DNA Master SYBR Green I kit (Roche (Penzberg, Germany)). The primer sets used in the study are shown in Table 1. Data were standardized using the GAPDH mRNA level. The melt curves for the PCR products were examined, and gene specific amplification was confirmed by the presence of a single band with the expected size in agarose gel electrophoresis.

TABLE 1

| | | | |
|---|---|---|---|
| | | Product | SEQ |
| | | size | ID |
| gene | primer sequence | (bp) | NO: |

| | | | | |
|---|---|---|---|---|
| 1110007F12Rik | Forward | 5′-GCCCTGTGCCTGATGTTCTAC-3′ | 111 | 5 |
| | Reverse | 5′-GCCCATGTCCTCCTTCCAC-3′ | | 6 |
| 2900064A13Rik | Forward | 5′-GTTTGACCCTGTCCGAGTCG-3′ | 205 | 7 |
| | Reverse | 5′-CGGGAGAACCATCATCATAACC-3′ | | 8 |
| Cc12 | Forward | 5′-TTAAAAACCTGGATCGGAACCAA-3′ | 121 | 9 |
| | Reverse | 5′-GCATTAGCTTCAGATTTACGGGT-3′ | | 10 |
| Cc19 | Forward | 5′-TCAGATTGCTGCCTGTCCTAT-3′ | 117 | 11 |
| | Reverse | 5′-GAACCCCCTCTTGCTGATAAAG-3′ | | 12 |
| Cxc15 | Forward | 5′-TGCGTTGTGTTTGCTTAACCG-3′ | 107 | 13 |
| | Reverse | 5′-AGCTATGACTTCCACCGTAGG-3′ | | 14 |
| Il1rn | Forward | 5′-GCTCATTGCTGGGTACTTACAA-3′ | 132 | 15 |
| | Reverse | 5′-CCAGACTTGGCACAAGACAGG-3′ | | 16 |
| I16 | Forward | 5′-TAGTCCTTCCTACCCCAATTTCC-3′ | 76 | 17 |
| | Reverse | 5′-TTGGTCCTTAGCCACTCCTTC-3′ | | 18 |

### Test Example 1

### Evaluation of HSC-Supporting Capacity of PA6 Subclones

Regarding the lack of the capability of supporting long-term hematopoiesis in vitro, PA6 subclones 2, 12, and 14 (hereunder referred to as S-2, S-12, and S-14) have been isolated, but the HSC-supporting capacity thereof had not specifically been evaluated (J Exp Med 176 (1992) 351-361). Hence, the present inventors evaluated the maintenance of HSC function after co-culture with PA6 subclone cells (S-2 and S-12) by in vitro CFC assay using CD34-KSL cells (Science 273 (1996) 242-245) as highly purified HSCs by the method of Example 1.4, and by in vivo competitive bone marrow repopulation assay by the method of Example 1.5. The murine bone marrow derived OP9 stromal cell line (Exp Hematol 22 (1994) 979-984), which is capable of supporting HSCs, served for control. As shown in Table 2, the CFC frequency decreased significantly after co-culture with PA6 S-2 and S-12 cells for 10 days compared with PA6 cells, with lower levels of engraftment and chimerism observed in the recipient mice. These results show that the PA6 subclone cells are substantially defective in the support of HSCs even after short-time co-culture.

TABLE 2
CFC frequency and competitive bone marrow repopulation ability

| | | Number (%) of mice | |
|---|---|---|---|
| | | with bone marrow | |
| Stromal cell | CFC frequency | repopulation | % chimerism |
| PA6 | 10.8 ± 7.51 | 6/9(66.7) | 13.2 ± 19.9 |
| PA6 S-2 | 0.60 ± 0.97 | 4/10(40.0) | 4.5 ± 11.2 |
| PA6 S-12 | 3.2 ± 3.05 | 3/8(37.5) | 3.4 ± 5.9 |
| OP9 | 28.2 ± 6.61 | 5/9(55.6) | 8.6 ± 11.5 |

A total of 80 CD34⁻KSL HSCs (10 cells/well) were co-cultured with stromal cells for 10 days, and then subjected to CFC assay. The CFC frequency is shown as a colony count per 10 inputs of CD34⁻KSL HSCs. In competitive bone marrow repopulation assay, 30 CD34⁻KSL HSCs were co-cultured with stromal cells for 10 days, and then transplanted to a mouse, previously exposed to a lethal dose of radiation. After 12 weeks of transplantation, peripheral blood cells from the recipient mouse were analyzed. The data shown indicate means for two separate experiments.

### Test Example 2

### Identification and Characterization of Genes Down-Regulated Specifically in PA6 Subclone Cells

### (1) Identification of Genes Downregulated Specifically in PA6 Subclone Cells

On the assumption that the causal gene for HSC support is downregulated in PA6 subclone cells, microarray analyses were performed on PA6 cells, PA6 S-2 and S-12 cells, as well as OP9 cells, by the method described in Example 1.6, using the Affymetrix GeneChip array, which comprises approximately 45,000 probe sets representing more than 34,000 mouse genes, in order to identify genes downregulated specifically in PA6 subclone cells. Gene expression profiling exhibited a slight difference in gene expression between PA6 cells and PA6 subclone cells. It was found that 144 genes were 2 folds or more down-regulated in both types of PA6 subclone cells, compared with PA6 cells. Of these downregulated genes, 41 were functionally unknown genes. Gene ontology analysis demonstrated that 65% of the known genes are membrane or extracellular space proteins. Expression was also observed in OP9 cells; eight genes with known or putative functions and three functionally unknown genes were selected for further analysis (Table 3).

TABLE 3
List of candidate genes downregulated in PA6 subclone cells, compared with PA6 cells

| | | | Microarray | | | | qPCR | | | |
|---|---|---|---|---|---|---|---|---|---|---|
| | | | vs. PA6 | | vs. OP9 | | vs. PA6 | | vs. OP9 | |
| | | | PA6 | PA6 | PA6 | PA6 | PA6 | PA6 | PA6 | PA6 |
| Gene symbol | Gene title | Ref Seq ID | S-2 | S-12 | S-2 | S-12 | S-2 | S-12 | S-2 | S-12 |
| 1110007F12Rik | RIKEN cDNA 1110007F12 | NM_197986 | 0.30 | 0.46 | 0.11 | 0.19 | 0.29 | 0.23 | 0.16 | 0.13 |
| | gene | | | | | | | | | |
| 1200009O22Rik | RIKEN cDNA 1200009O22 | NM_025817 | 0.30 | 0.21 | 2.79 | 1.48 | ND | ND | ND | ND |
| | gene | | | | | | | | | |
| 2900064A13Rik | RIKEN cDNA 2900064A13 | NM_133749 | 0.41 | 0.37 | 1.23 | 1.16 | 0.29 | 0.33 | 0.48 | 0.55 |
| | gene | | | | | | | | | |
| Ccl2 | Chemokine (C—C motif) | NM_011333 | 0.09 | 0.13 | 0.04 | 0.06 | 0.16 | 0.07 | 0.09 | 0.04 |
| | ligand 2 | | | | | | | | | |
| Ccl9 | Chemokine (C—C motif) | NM_011338 | 0.14 | 0.19 | 0.03 | 0.04 | 0.10 | 0.09 | 0.04 | 0.03 |
| | ligand 9 | | | | | | | | | |
| Cd53 | CD53 antigen | NM_007651 | 0.23 | 0.15 | 0.28 | 0.14 | ND | ND | ND | ND |
| Cxcl5 | Chemokine (C—X—C | NM_009141 | 0.33 | 0.23 | 0.43 | 0.38 | 0.20 | 0.13 | 0.90 | 0.60 |
| | motif) ligand 5 | | | | | | | | | |
| Hgf | hepatocyte growth | NM_010427 | 0.41 | 0.41 | 0.85 | 0.88 | ND | ND | ND | ND |
| | factor | | | | | | | | | |
| Il1rn(IL-1rn) | Interleukin 1 | NM_031167 | 0.09 | 0.12 | 0.26 | 0.31 | <0.03 | 0.04 | <1.25 | 1.45 |
| | receptor antagonist | | | | | | | | | |
| Il6(IL-6) | Interleukin 6 | NM_031168 | 0.38 | 0.44 | 2.07 | 2.62 | 0.30 | 0.16 | 2.42 | 1.31 |
| Ppap2b | phosphatidic acid | NM_080555 | 0.22 | 0.22 | 0.09 | 0.09 | ND | ND | ND | ND |
| | phosphatase type | | | | | | | | | |

These are data obtained by microarray and quantitative real time PCR (qPCR) analyses; relative expression levels were calculated in PA6 subclones (S-2 and S-14), compared with PA6 and OP9 cells. The data shown indicate means of changes in two separate experiments. ND: not determined.

### (2) Characterization of Candidate Genes

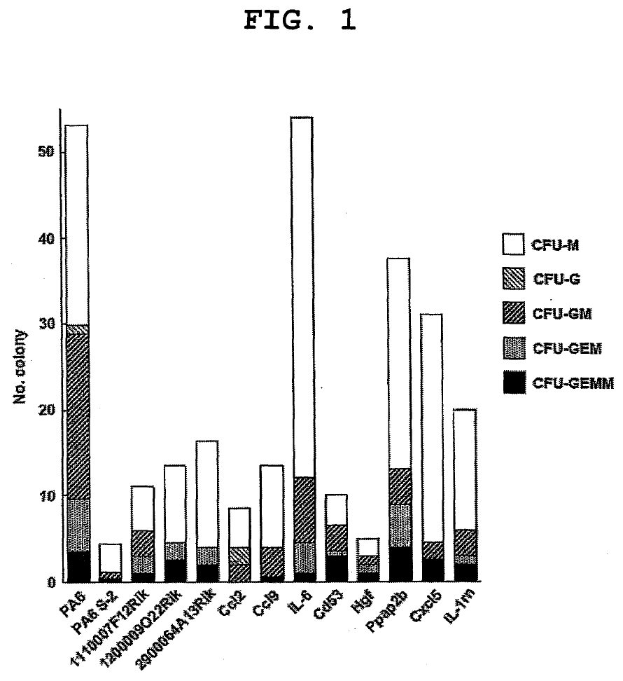

To analyze candidate genes supposed to confer the HSC-supporting activity of PA6 cells, lentivirus vectors harboring corresponding cDNAs were infected to PA6 S-2 cells. In all experiments, judging from the ratio of Venus-positive cells as determined by FACS analysis, transduction efficiency was 90% or more. CD34⁻KSL HSCs were co-cultured with PA6 S-2 cells expressing each candidate gene for 10 days, and then subjected to CFC assay. As shown in FIG. 1, when CD34⁻KSL HSCs were co-cultured with PA6 S-2 cells overexpressing a candidate gene other than Hgf, the CFC frequency increased, compared with PA6 S-2 cells. Morphological analysis of the colonies demonstrated that the number of CFU-GEMMs, the most primitive colony type observed in CFC assay, increased, except for Cc12 and Cc19. Noticeably, overexpression of IL-6 resulted in an increase in colony count to the same level as that with PA6 cells, but the majority (about 80%) of the colonies were CFU-M.

The present inventors then performed competitive bone marrow repopulation assay, as described in Example 1.5, to determine the effects of overexpression of seven candidate genes including Cc19, IL-6, Ppap2b, Cxc15, and three unidentified genes. As shown in Table 4, reconstruction of donor cell-derived hematopoiesis was observed in all mice transplanted with HSCs co-cultured with PA6 S-2 cells overexpressing 1200009022Rik or IL-6 and PA6 cells. However, a comparison of chimerism did not reveal a significant difference from PA6 S-2 cells; it was suggested that overexpression of 1200009022Rik or IL-6 might not cause a complete recovery from the lack of the HSC-supporting activity of PA6 S-2 cells. Microarray analysis showed that IL-6 expression was about 2 fold upregulated in PA6 S-2 and S-12 cells, compared with OP9 cells, and this was confirmed by quantitative real time PCR analysis (Table 3). It has also been demonstrated that IL-6 is unable to maintain CD34⁻KSL HSCs when used alone or in combination with SCF (J Exp Med 192 (2000) 1281-1288). IL-6 is involved in various processes in hematopoiesis, and has been used to expand HSCs ex vivo (Annu Rev Immunol 15 (1997) 797-819); however, IL-6 may not be important for the maintenance of HSCs by stromal cells.

TABLE 4

| | Number (%) of mice | | | | |
|---|---|---|---|---|---|
| | with bone marrow | % chimerism | | | |
| | reconstitution | Total | Myeloid | B lymphoid | T lymphoid |
| PA6 | 9/9(100) | 22.1 ± 25.1 | 30.9 ± 30.9 | 25.1 ± 26.5 | 17.4 ± 23.0 |
| PA6 S-2 | 4/10(40) | 7.3 ± 14.0 | 4.0 ± 7.4 | 10.8 ± 18.8 | 9.2 ± 17.8 |
| 1110007F12Rik | 7/10(70) | 16.2 ± 14.8* | 21.8 ± 26.3 | 18.7 ± 16.9 | 15.5 ± 16.7 |
| 1200009O22Rik | 8/8(100) | 8.2 ± 10.2 | 14.5 ± 12.2 | 9.6 ± 13.4 | 8.3 ± 11.3 |
| 2900064A13Rik | 2/5(40) | 3.3 ± 4.6 | 10.5 ± 20.6 | 3.6 ± 5.4 | 1.0 ± 2.2 |
| Ccl9 | 2/5(40) | 11.1 ± 15.0 | 14.2 ± 19.5 | 12.7 ± 17.6 | 11.4 ± 15.7 |
| IL-6 | 9/9(100) | 8.5 ± 13.5 | 8.1 ± 7.1 | 11.6 ± 16.9 | 9.2 ± 16.3 |
| Ppap2b | 8/10(80) | 9.7 ± 10.4 | 20.5 ± 26.7 | 9.2 ± 9.7 | 9.6 ± 14.6 |
| Cxcl5 | 3/5(60) | 3.0 ± 4.0 | 12.8 ± 23.6 | 2.5 ± 3.5 | 1.9 ± 1.8 |

A total of 30 CD34⁻KSL HSCs were co-cultured with PA6 cells, PA6 S-2 cells, or PA6 S-2 cells that express any of the indicated genes for 10 days, and then transplanted to a mouse, previously exposed to a lethal dose of radiation. After 16 weeks of transplantation, peripheral blood cells from the recipient mouse were analyzed. Each data indicates means ± SD.
*P = 0.18, Student's t-test.

PA6 S-2 cells that overexpressed 2900064A13Rik, CC19, Ppap2b, or Cxc15 did not exhibit a definite effect on HSC-supporting activity. By contrast, overexpression of 1110007F12Rik tended to increase both the frequency of engrafted mice and chimerism. To confirm the effect of 1110007F12Rik expression on the supporting activity of PA6 cells, the present inventors performed the experiments shown below. First, compared with PA6 and OP9 cells, downregulation of 1110007F12Rik expression in PA6 S-2 and S-12 cells was confirmed by quantitative real time PCR analysis (Table 3). Subsequently, PA6 S-2 cells were infected with a lentivirus vector expressing 1110007F12Rik with a myc tag, and the expression thereof was detected by Western blotting and immunofluorescence staining with an anti-myc antibody. Co-culture with PA6 S-2 cells overexpressing 1110007F12Rik with a myc tag resulted in an increase in the CFC frequency to the same level as that shown in FIG. 1 (data not shown).

These findings demonstrated a partial recovery from the lack of the HSC maintenance activity of PA6 S-2 cells by the 1110007F12Rik protein, showing that the protein is required for the maintenance and expansion of HSCs.

Furthermore, the present inventors cloned the human homologs of the 1110007F12Rik gene from HeLa cells and 293T cells according to a method known in the art and using the PCR method. The base sequence cloned from HeLa cells is shown in SEQ ID NO: 19, and the amino acid sequence encoded by this base sequence is shown in SEQ ID NO: 20. The base sequence cloned from 293T cells is shown in SEQ ID NO: 21, and the amino acid sequence encoded by this base sequence is shown in SEQ ID NO: 22.

### INDUSTRIAL APPLICABILITY

According to the present invention, hematopoietic stem cells can be maintained and expanded ex vivo, and it is possible to efficiently supply stem cells required for hematopoietic stem cell transplantation, and to solve the current problems, including the shortage of donors and risks on the donor.

This application is based on a patent application No. 2007-341392 filed in Japan, the contents of which are incorporated in full herein by this reference.

[Sequence Listing]

1. An agent for maintaining or expanding hematopoietic stem cells, comprising a protein comprising the same or substantially the same amino acid sequence as an amino acid sequence shown by SEQ ID NO:2 or 4.

2. An agent that confers hematopoiesis-supporting capacity to cells, comprising an expression vector harboring a nucleic acid that encodes a protein comprising the same or substantially the same amino acid sequence as an amino acid sequence shown by SEQ ID NO:2 or 4.

3. An agent for maintaining or expanding hematopoietic stem cells, comprising a mammalian cell incorporating an expression vector harboring a nucleic acid that encodes a protein comprising the same or substantially the same amino acid sequence as an amino acid sequence shown by SEQ ID NO:2 or 4.

4. The agent according to claim 3, wherein the mammalian cell is a stromal cell.

5. The agent according to claim 3, wherein the mammalian cell does not possess hematopoiesis-supporting capacity.

6. The agent according to claim 3, wherein the expression vector is a lentivirus vector or a retrovirus vector.

7. The agent according to claim 4, wherein the expression vector is a lentivirus vector or a retrovirus vector.

8. The agent according to claim 5, wherein the expression vector is a lentivirus vector or a retrovirus vector.

9. A method of producing a mammalian cell with improved hematopoiesis-supporting capacity, comprising introducing to a mammalian cell an expression vector harboring a nucleic acid that encodes a protein comprising the same or substantially the same amino acid sequence as an amino acid sequence shown by SEQ ID NO:2 or 4, and selecting a cell that expresses the nucleic acid.

10. A method of maintaining or expanding hematopoietic stem cells, comprising culturing hematopoietic stem cells in the presence of a protein comprising the same or substantially the same amino acid sequence as an amino acid sequence shown by SEQ ID NO:2 or 4.

11. The method according to claim 10, comprising culturing hematopoietic stem cells in the co-presence of at least one kind of cytokine.

12. A method of maintaining or expanding hematopoietic stem cells, comprising co-culturing a mammalian cell incorporating an expression vector harboring a nucleic acid that encodes a protein comprising the same or substantially the same amino acid sequence as an amino acid sequence shown by SEQ ID NO:2 or 4, and a hematopoietic stem cell.

13. The method according to claim 12, wherein the mammalian cell is a stromal cell.

14. The method according to claim 12, wherein the mammalian cell does not possess hematopoiesis-supporting capacity.

15. The method according to claim 12, wherein the expression vector is a lentivirus vector or a retrovirus vector.

16. The method according to claim 13, wherein the expression vector is a lentivirus vector or a retrovirus vector.

17. The method according to claim 14, wherein the expression vector is a lentivirus vector or a retrovirus vector.

18. A cell population comprising expanded hematopoietic stem cells, which is obtained by the method according to claim 10.

19. A cell population comprising expanded hematopoietic stem cells, which is obtained by the method according to claim 12.

20. An agent for ameliorating hematopoietic function, comprising the cell population according to claim 18.

21. An agent for ameliorating hematopoietic function, comprising the cell population according to claim 19.


SEQUENCE LISTING (22 sequences)

SEQ ID NO: 1 - DNA, 558 bp, Homo sapiens
  CDS (1)..(558)
atggccggcccaaggcctcggtggcgcgaccagctgctgttcatgagcatcatagtcctcgtgattgtggtcatctgcctgatgttttacgctcttctctgggaggctggcaacctcactgacctgcccaacctgagaatcggcttctataacttctgcctgtggaatgaggacaccagcaccctacagtgtcaccagttccctgagctggaagccctgggggtgcctcgggttggcctgggcctggccaggcttggcgtgtacgggtccctggtcctcaccctctttgccccccagcctctcctcctagcccagtgcaacagtgatgagagagcgtggcggctggcagtgggcttcctggctgtgtcctctgtgctgctggcaggcggcctgggcctcttcctctcctatgtgtggaagtgggtcaggctctccctcccggggcctgggtttctagctctgggcagcgcccaggccttactcatcctcttgcttatagccatggctgtgttccctctgagggctgagagggctgagagcaagcttgagagctgctaa

SEQ ID NO: 2 - protein, 185 aa, Homo sapiens
MAGPRPRWRDQLLFMSIIVLVIVVICLMFYALLWEAGNLTDLPNLRIGFYNFCLWNEDTSTLQCHQFPELEALGVPRVGLGLARLGVYGSLVLTLFAPQPLLLAQCNSDERAWRLAVGFLAVSSVLLAGGLGLFLSYVWKWVRLSLPGPGFLALGSAQALLILLLIAMAVFPLRAERAESKLESC

SEQ ID NO: 3 - DNA, 558 bp, Mus musculus
  CDS (1)..(558)
atggccttctccaggctgtggcggaacaaccacctgcccttcgtgggcatcatgatcctcttggccgcggccctgtgcctgatgttctacgctctcctctggaaggctggcaacctcgctgacctacctagcctgagaatcggcttctacaacttctgtctgtggaaggaggacatgggctccctagcatgttacaactttcccgagctggacgtgctgggcattcctcaggttggactagccctggccaggctcggtgtgtatggagccctggtcctcacagtgttcgtccctctgcctctcctccttgcccagtacaacagagatgagggagagtggcggctggccgtgtgcttcctggcggcatcctccattctgttggccggaggactaagcctcttcctctccttcgtgtggaagtggctcaggctctccttcctggggcctgccttgccagctctatgcctagcccagctgttactcatcttcttgcttgtggccacggttaggttcccgccacgggataaggaggacaagaaccagtgggagaactgttag

SEQ ID NO: 4 - protein, 185 aa, Mus musculus
MAFSRLWRNNHLPFVGIMILLAAALCLMFYALLWKAGNLADLPSLRIGFYNFCLWKEDMGSLACYNFPELDVLGIPQVGLALARLGVYGALVLTVFVPLPLLLAQYNRDEGEWRLAVCFLAASSILLAGGLSLFLSFVWKWLRLSFLGPALPALCLAQLLLIFLLVATVRFPPRDKEDKNQWENC

SEQ ID NO: 5 - DNA, 21 bp, Artificial
  forward primer
gccctgtgcctgatgttctac

SEQ ID NO: 6 - DNA, 19 bp, Artificial
  reverse primer
gcccatgtcctccttccac

SEQ ID NO: 7 - DNA, 20 bp, Artificial
  forward primer
gtttgaccctgtccgagtcg

SEQ ID NO: 8 - DNA, 22 bp, Artificial
  reverse primer
cgggagaaccatcatcataacc

SEQ ID NO: 9 - DNA, 23 bp, Artificial
  forward primer
ttaaaaacctggatcggaaccaa

SEQ ID NO: 10 - DNA, 23 bp, Artificial
  reverse primer
gcattagcttcagatttacgggt

SEQ ID NO: 11 - DNA, 21 bp, Artificial
  forward primer
tcagattgctgcctgtcctat

SEQ ID NO: 12 - DNA, 22 bp, Artificial
  reverse primer
gaaccccctcttgctgataaag

SEQ ID NO: 13 - DNA, 21 bp, Artificial
  forward primer
tgcgttgtgtttgcttaaccg

SEQ ID NO: 14 - DNA, 21 bp, Artificial
  reverse primer
agctatgacttccaccgtagg

SEQ ID NO: 15 - DNA, 22 bp, Artificial
  forward primer
gctcattgctgggtacttacaa

SEQ ID NO: 16 - DNA, 21 bp, Artificial
  reverse primer
ccagacttggcacaagacagg

SEQ ID NO: 17 - DNA, 23 bp, Artificial
  forward primer
tagtccttcctaccccaatttcc

SEQ ID NO: 18 - DNA, 21 bp, Artificial
  reverse primer
ttggtccttagccactccttc

SEQ ID NO: 19 - DNA, 558 bp, Artificial
  HeLa cell
atggccggcccaaggcctcggtggcgcgaccagctgctgttcatgagcatcatagtcctcgtgattgtggtcatctgcctgatgttatacgctcttctctgggaggctggcaacctcactgacctgcccaacctgagaatcggcttctataacttctgcctgtggaatgaggacaccagcaccctacagtgtcaccagttccctgagctggaagccctgggggtgcctcgggttggcctgggcctggccaggcttggcgtgtacgggtccctggtcctcaccctctttgccccccagcctctcctcctagcccagtgcaacagtgatgagagagcgtggcggctggcagtgggcttcctggctgtgtcctctgtgctgctggcaggcggcctgggcctcttcctctcctatgtgtggaagtgggtcaggctctccctcccggggcctgggtttctagctctgggcagcgcccaggccttactcatcctcttgcttatagccatggctgtgttccctctgagggctgagagggctgagagcaagcttgagagctgctaa

SEQ ID NO: 20 - protein, 185 aa, Artificial
  Synthetic Construct
MAGPRPRWRDQLLFMSIIVLVIVVICLMLYALLWEAGNLTDLPNLRIGFYNFCLWNEDTSTLQCHQFPELEALGVPRVGLGLARLGVYGSLVLTLFAPQPLLLAQCNSDERAWRLAVGFLAVSSVLLAGGLGLFLSYVWKWVRLSLPGPGFLALGSAQALLILLLIAMAVFPLRAERAESKLESC

SEQ ID NO: 21 - DNA, 558 bp, Artificial
  293T cell
atggccggcccaaggcctcagtggcgcgaccagctgctgttcatgagcatcatagtcctcgtgattgtggtcatctgcctgatgttatacgctcttctctgggaggctggcaacctcactgacctgcccaacctgagaatcggcttctataacttctgcctgtggaatgaggacaccagcaccctacagtgtcaccagttccctgagctggaagccctgggggtgcctcgggttggcctgggcctggccaggcttggcgtgtacgggtccctggtcctcaccctctttgccccccagcctctcctcctagcccagtgcaacagtgatgagagagcgtggcggctggcagtgggcttcctggctgtgtcctctgtgctgctggccggcggcctgggcctcttcctctcctatgtgtggaagtgggtcaggctctccctcccggggcctgggtttctagctctgggcagcgcccaggccttactcatcctcttgcttatagccatggctgtgttccctctgagggctgagagggctgagagcaagcttgagagctgctaa

SEQ ID NO: 22 - protein, 185 aa, Artificial [sequence omitted]